(19) United States
(12) Patent Application Publication
(10) Pub. No.: US 2022/0136036 A1
(43) Pub. Date: May 5, 2022

(54) METHODS AND SYSTEMS OF PCR-BASED RECOMBINANT ADENO-ASSOCIATED VIRUS MANUFACTURE

(71) Applicant: APDN (B.V.I) Inc., Tortola (VG)

(72) Inventors: Michael E. HOGAN, Stony Brook, NY (US); Stephen HUGHES, Port Jefferson Station, NY (US); Yuhua SUN, Stony Brook, NY (US)

(73) Assignee: APDN (B.V.I) Inc., Tortola (VG)

(21) Appl. No.: 17/578,004

(22) Filed: Jan. 18, 2022

Related U.S. Application Data
(63) Continuation-in-part of application No. 16/988,025, filed on Aug. 7, 2020.
(60) Provisional application No. 62/916,333, filed on Oct. 17, 2019.
(60) Provisional application No. 62/883,701, filed on Aug. 7, 2019.

(51) Int. Cl. C12Q 1/686; C12Q 1/6848; C12N 7/00; C12N 15/86; C12Q 1/6883

(52) U.S. Cl. CPC C12Q 1/686; C12Q 1/6848; C12N 7/00; C12N 2750/14152; C12Q 1/6883; C12N 2750/14143; C12N 2750/14122; C12N 15/86

(57) ABSTRACT

The invention relates to methods of treating diseases comprising administering to a subject a composition comprising the recombinant adeno-associated virus (rAAV). The rAAV is produced by a method comprising: obtaining a template DNA sequence containing a [ITR-cargo-ITR] DNA sequence motif; designing a PCR primer pair such that the 3′ terminus of both the forward and reverse PCR primers overlap only about the last 2-8 bases of the A/A′ ITR sequences and the 5′ terminus of both the forward and reverse PCR primers extend into about 20-35 bases of the flanking sequences; performing PCR with cycling parameters comprising a combined annealing/extension step at a temperature greater than 70° C., thereby producing a plurality of amplicon polynucleotides containing the desired [ITR-cargo-ITR] DNA sequence motif; transfecting the amplicon polynucleotides containing the desired [ITR-cargo-ITR] DNA sequence motif into a packaging cell line; and purifying the lysed cells to collect a quantity of rAAV.

### CROSS REFERENCE TO RELATED APPLICATIONS

The present application claims the benefit of U.S. patent application Ser. No. 16/988,025, filed on Aug. 7, 2020; U.S. provisional patent application No. 62/883,701, filed on Aug. 7, 2019; and U.S. provisional patent application No. 62/916,333, filed on Oct. 17, 2019; the contents of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Sep. 1, 2020, is named 189542 SL.txt and is 1,379 bytes in size.

### BACKGROUND OF THE INVENTION

### 1. Technical Field

The present invention relates to systems and methods to produce recombinant adeno-associated virus (rAAV) utilizing one or more DNA constructs manufactured via polymerase chain reaction (PCR).

### 2. Background of the Invention

The manufacture of large quantities of high-quality DNA is currently a major bottleneck in the production of viral vectors utilized in, among other things, gene therapy and vaccines. Currently, bacterial plasmids, which are small circular episomal DNA molecules that can replicate independently of bacterial chromosomal DNA, are utilized as the primary source of DNA to produce viral vectors. In addition to long amplification times, measured in days or weeks, the amplification of DNA via bacterial plasmids for use in viral vector manufacture has additional drawbacks such as the necessity of complex and expensive purification steps, the risk of endotoxin contamination, antibiotic resistance gene transfer, other plasmid derived DNA sequence transfers, as well as challenges with integration into robotic and/or automated workflows. Moreover, certain DNA sequences that are necessary to produce specific viral vectors (e.g. inverted tandem repeats) are ill-suited for plasmid-based amplification and lead to high failure rates and low viral titer.

One of the most promising viral vectors is adeno-associated virus (AAV), which, in most instances, is manufactured by triple transfection of plasmid DNA constructs into packaging cell lines to produce recombinant adeno-associated virus (rAAV). rAAV manufacture requires three different DNA constructs that must be transfected into a packaging cell line. These DNA constructs are: (i) a DNA construct containing the AAV Rep and Cap genes required for capsid formation and replication (“rep/cap”); (ii) a DNA construct containing the necessary adenovirus helper genes (“AAV helper”); and (iii) a DNA construct containing the cargo (transgene) of interest flanked on both sides by inverted terminal repeats (ITRs) (“[ITR-cargo-ITR]”). These three DNA constructs are currently amplified and supplied to rAAV manufacturing facilities in the form of DNA plasmids.

The ITR DNA sequence of AAV has emerged as an enabling element for rAAV based therapeutics, as any transgene which is to be delivered by a rAAV therapy must be flanked on each side by a single copy of the 145 bp long ITR sequence. Direct proximity of the cargo of interest to the ITR regions is an absolute requirement for successful manufacture of rAAV based therapies, as the ITR regions must be present for successful packaging of the transgene into the viral capsid. Without proper flanking ITR sequences, rAAV will not package the desired transgene (cargo) and the resultant rAAV therapy will fail.

Until now, the three DNA constructs necessary for rAAV production have been manufactured via bacterial plasmid-based systems. Recently, due to concerns about bacterial plasmid safety in therapeutics and the operational challenges created by the use of plasmid-based DNA amplification systems, it has become important to eliminate the use of bacterial plasmids to produce one or more of the DNA constructs necessary to manufacture rAAV. Heretofore, it was believed in the art that scalable and accurate PCR-based amplification of the [ITR-cargo-ITR] construct was not possible due to the unique secondary structures of the ITR regions that are ill suited for PCR-based amplification.

In addition, for certain therapeutic applications, rAAV vectors consisting exclusively or predominantly of single stranded DNA (ssDNA) of a single polarity can lead to higher viral titers and greater efficacy of a resultant therapeutic. ssDNA of a single polarity may be the positive (sense) or reverse/minus (anti-sense) polarity of the rAAV ssDNA genome. Herein, systems and methods of creating single polarity rAAV vectors produced via PCR-based manufacturing of specialized [ITR-cargo-ITR] amplicons are disclosed.

The invention of the instant application discloses novel methods and systems for the PCR-based manufacture of the DNA constructs necessary for rAAV production, including the [ITR-cargo-ITR] construct. In addition, the methods and systems of the instant application can also be adopted to produce rAAV vectors packing a single polarity of its ssDNA genome via the use of specialized [ITR-cargo-ITR] amplicons.

### BRIEF SUMMARY OF THE INVENTION

The present invention relates to systems and methods to produce recombinant adeno-associated virus (rAAV) utilizing one or more DNA constructs produced via polymerase chain reaction (PCR).

In one aspect, the invention provides a method for amplifying a DNA sequence comprising the general sequence structure of [ITR-cargo-ITR] wherein: (i) the 3′ terminus of both the forward and reverse PCR primer pair is designed to overlap only the last 2-8 bases of the “A” ITR sequence; (ii) the 5′ end of each PCR primer extends into about 20-35 bases of the flanking DNA sequence adjacent to the ITR sequences; (iii) the PCR cycling parameters have a combined annealing/extension step at a temperature greater than 70° C.; and (iv) the PCR master mix contains one or more osmolytes. In some embodiments, the osmolyte may be betaine. In another aspect, the DNA flanking sequences are designed for high-affinity PCR primer binding.

In another aspect, a method of manufacturing amplicon polynucleotides containing the sequence motif [ITR-cargo-ITR] via CPR is provided, said method comprising: (i) obtaining a desired template DNA sequence containing a [ITR-cargo-ITR] DNA sequence motif; (ii) designing a PCR primer pair such that the 3′ terminus of both the forward and reverse PCR primers overlap only about the last 2-8 bases of the A and A′ ITR sequences and the 5′ terminus of both the forward and reverse PCR primers extend into about 20-35 bases of the flanking DNA sequences adjacent to the ITR sequences; (iii) performing a PCR amplification reaction with PCR cycling parameters comprising a combined annealing/extension step at a temperature greater than 70° C., wherein the PCR amplification reaction contains on or more osmolytes, thereby producing a plurality of amplicon polynucleotides containing the desired DNA sequence motif [ITR-cargo-ITR]. In preferred embodiments, the osmolyte is betaine. The template DNA sequence containing a [ITR-cargo-ITR] DNA sequence motif may be obtained from a plasmid or from a non-plasmid source such as a DNA construct assembled with solid-state syntheses or other polynucleotide manufacturing process. The resultant plurality of amplicon polynucleotides containing the desired DNA sequence motif [ITR-cargo-ITR] may or may not be sequence verified via next generation sequencing. A representative sample of the amplicon polynucleotides containing the desired DNA sequence motif [ITR-cargo-ITR] may also be verified via next generation sequencing.

In another aspect, a method for the production of recombinant adeno-associated virus (rAAV) is disclosed, said method comprising; (i) obtaining a desired template DNA sequence containing a [ITR-cargo-ITR] DNA sequence motif; (ii) designing a PCR primer pair such that the 3′ terminus of both the forward and reverse PCR primers overlap only about the last 2-8 bases of the A and A′ ITR sequences and the 5′ terminus of both the forward and reverse PCR primers extend into about 20-35 bases of the flanking DNA sequences adjacent to the ITR sequences; (iii) performing a PCR amplification reaction with PCR cycling parameters comprising a combined annealing/extension step at a temperature greater than 70° C., wherein the PCR amplification reaction contains on or more osmolytes, thereby producing a plurality of amplicon polynucleotides containing the desired sequence motif [ITR-cargo-ITR]; (iv) obtaining a quantity of the AAV rep/cap DNA sequence; (v) obtaining a quantity of AAV helper DNA sequence; (vi) transfecting the amplicon polynucleotides containing the desired sequence motif [ITR-cargo-ITR], the AAV rep/cap DNA sequence and the AAV helper DNA sequence into a packaging cell line; (vii) after cell line expansion, lysing and purifying the lysed cells to collect a quantity of rAAV.

In yet another aspect, through use of forced asymmetrical PCR or ITR modification, rAAV vectors packaging a single polarity of its ssDNA genome can be manufactured via the use of specialized [ITR-cargo-ITR] amplicon polynucleotides.

### BRIEF DESCRIPTION OF THE FIGURES

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 is a wild-type ITR DNA sequence (SEQ ID NO: 4) showing the ITR secondary structure, the A, B, C and D elements, and the location of primer binding according to an embodiment of the invention.

FIG. 2 is a plasmid map of a template [ITR-GFP-ITR] showing primer locations according to an embodiment of the invention.

FIG. 3 is an illustration of the primer design principle according to an embodiment of the invention.

FIG. 4 is a flow diagram of an embodiment of the system and method to manufacture single polarity rAAV vectors via the use of specialized [ITR-cargo-ITR] amplicons.

FIG. 5 is an electropherogram showing DNA amplicon characteristics as produced according to an embodiment of the invention.

FIG. 6 is an electropherogram showing DNA amplicon characteristics as produced according to an embodiment of the invention.

FIG. 7 is an electropherogram showing DNA amplicon characteristics as produced according to an embodiment of the invention.

FIG. 8 is an electropherogram showing DNA amplicon characteristics as produced according to an embodiment of the invention.

### DETAILED DESCRIPTION OF THE INVENTION

The following documentation provides a detailed description of exemplary embodiments of the invention. Although a detailed description as provided herein contains many specifics for the purposes of illustration, anyone of ordinary skill in the art will appreciate that many variations, equivalents and alterations to the following details are within the scope of the invention. Accordingly, the following preferred embodiments of the invention are set forth without any loss of generality to, and without imposing limitations upon, the claimed invention. Thus, the scope of the invention should be determined by the appended claims and their legal equivalents, and not merely by the preferred examples or embodiments given herein.

### Definitions

The term “amplicon” as used herein means a DNA or RNA polynucleotide that is the product of an enzymatic or chemical based amplification event or reaction. Amplification events or reactions include, without limitation, the polymerase chain reaction (PCR), loop mediated isothermal amplification, rolling circle amplification, nucleic acid sequence base amplification, and ligase chain reaction or recombinase polymerase amplification. An amplicon may be comprised of single stranded and/or double stranded DNA, and/or a combination thereof. An amplicon cannot be produced by or be the product of bacterial plasmid propagation within bacteria.

The term “continuous flow PCR device” means a PCR device as disclosed in U.S. Pat. Nos. 8,293,471, 8,986,982 and 8,163,489.

The term “episomal” means a DNA polynucleotide that replicates independently from chromosomal DNA. Episomal DNA may reside in a cell's nucleus.

The term “expression” refers to the transcription and/or translation of an expression cassette.

The term “expression cassette” means a nucleic acid sequence consisting of one or more genes and the sequences controlling their expression. At a minimum, an expression cassette shall include a promoter (or other expression control sequence) and an open reading frame (ORF).

The term “expression control sequence” means a nucleic acid sequence that directs transcription of a nucleic acid and/or open reading frame. An expression control sequence can be a promotor or an enhancer.

The term a “subject” is any mammal, including without limitation humans, monkeys, farm animals, pets, horses, dogs and cats. In an exemplary embodiment, the subject is human.

The term “next generation sequencing” (NGS) includes any form of high-throughput DNA or RNA sequencing. This includes, without limitation, sequencing by synthesis, sequencing by ligation, nanopore sequencing, single-molecule real-time sequencing and ion semiconductor sequencing.

The term “transfection” means the uptake of exogenous or heterologous RNA or DNA by a cell. Without limitation, transfection may be accomplished by direct uptake, electroporation, chemical or other substance-based methods (e.g. calcium chloride, rubidium chloride, alcohol, DEAE-dextran, PEI) lipofection, soluporation, cationic liposomes, cationic polymers, lipoplexes, synthetic branched dendrimers, microprojectile bombardment, cellular surgery, lipid nanoparticles (LNPs), and/or viral transduction.

The term “large-scale PCR” means a PCR reaction wherein the total PCR reaction volume is greater than 0.7 liters. Large-scale PCR may be performed in a single reaction vessel or may be performed in a plurality of reaction vessels simultaneously.

The term “cargo” means one or more expression cassettes. Cargo, may be, without limitation, a transgene.

The term “transgene” means a gene, genetic material or other expression cassette that is artificially introduced into the genome of a subject.

The term “ITR” means inverted terminal repeat DNA sequence. The ITR sequence may be wild-type and comprise 145 bases each. The ITR sequence may also be modified and may be comprised of more or less than 145 bases. The ITR may be comprised of wild-type A, B, C and D elements, or one or more of said elements may be modified.

The term “[ITR-cargo-ITR]” means a DNA sequence comprised of the general motif of a cargo (transgene) flanked on both sides by an ITR sequence. A [ITR-cargo-ITR] is flanked on either side by a flanking sequence.

The use of the alternative (e.g., “or”) should be understood to mean either one, both, or any combination thereof of the alternatives. As used herein, the indefinite articles “a” or “an” should be understood to refer to “one or more” of any recited or enumerated component.

### PCR Amplification of the [ITR-Cargo-ITR] DNA Construct

The two ITR sequences that flank the transgene cargo in the important [ITR-cargo-ITR] DNA construct necessary for rAAV manufacture are poorly compatible with ordinary methods of PCR-based production. This poor compatibility stems from the structure of the ITR sequence domain, rather than its proximity to the transgene.

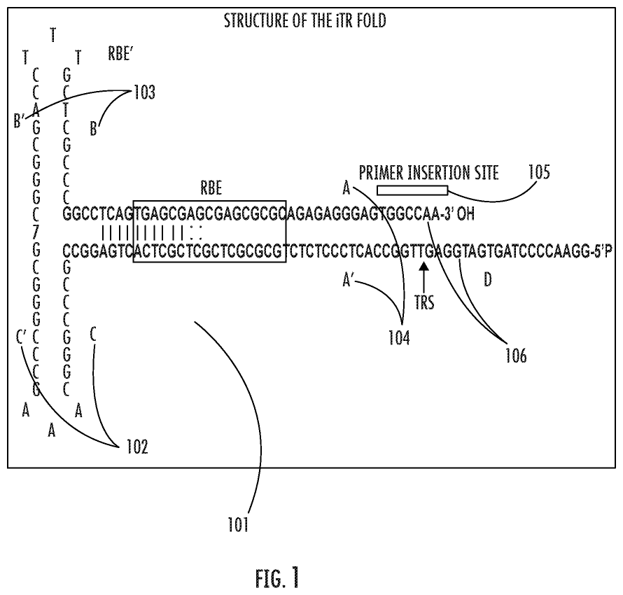

As shown in FIG. 1, the ITR sequence (101) is extremely G-C rich and contains multiple self-complementary sequences A/A′ (104) BB′ (103) and C/C′ (102) that allow the single stranded version of the ITR sequence to fold into a very stable stem-loop secondary structure (101). Both ITR sequences are flanked by flanking sequences (106) that may or may not form secondary structures. These features of the ITR, which are necessary for successful rAAV production, are problematic for conventional PCR-based amplification, which struggles with both G-C rich sequences and secondary structures. Under conventional PCR-based amplification methodologies, upon the first heat denaturation step in the PCR reaction, a dsDNA template containing an ITR region is denatured to form the corresponding ssDNA template, which upon cooling, is driven by the presence of self-complementary G-C rich regions (102, 103 and 104) to fold into the highly stable hairpin secondary structure (101) shown in FIG. 1, which serves to block proper PCR primer (105) binding to the template necessary to initiate PCR amplification and, subsequently, the extension of the bound primer through the highly folded template's secondary structure. The result is: (i) complete failure to amplify the [ITR-cargo-ITR] construct; (ii) very low amplification yield of the [ITR-cargo-ITR] construct; and/or (iii) amplification of the [ITR-cargo-ITR] construct resulting in one or more undesired side reactions producing additional amplicons.

Embodiments of the systems and methods of the present invention address these issues with novel systems and methods for the PCR-based amplification of the [ITR-cargo-ITR]. In an embodiment, a [ITR-cargo-ITR] DNA construct may be successfully amplified via PCR by utilizing the following PCR modifications in conjunction: (i) PCR primers designed for calculated minimal insertion into the ITR fold of between 2 and 10 bases in the area of the A/A′ (104); (ii) extension of the 5′ end of the PCR primer into about 20 bases to 30 bases of ITR flanking DNA sequence (106) such that the forward and reverse PCR primers bind to the flanking regions with high affinity and with minimal insertion into the ITR fold of between 2 and 10 bases; (iii) use of high annealing temperature based two-step PCR; and (iv) the introduction of an osmolyte into the PCR reaction buffer. The PCR primers may also be designed for calculated minimal insertion into the ITR fold of between 2 and 8 bases; 2 and 6 bases; and 2 and 5 bases.

In an embodiment, a [ITR-cargo-ITR] DNA construct is PCR amplified according to the following method: (i) design and assembly of forward and reverse PCR primers that bind to the ITR flanking regions of a [ITR-cargo-ITR] construct (106), wherein the 3′terminus of said forward and reverse PCR primers minimally insert into the ITR fold between 2 and 8 bases in the area of A/A′ (104) when bound to flanking regions and wherein the 5′ terminus of said forward and reverse PCR primers extend into about 20-35 bases of the flanking region DNA sequences (106); (ii) the inclusion of betaine or other osmolyte into the PCR reaction composition; and (iii) the utilization of 2-step PCR with a combined annealing/extension temperature greater than 70.degree. C.

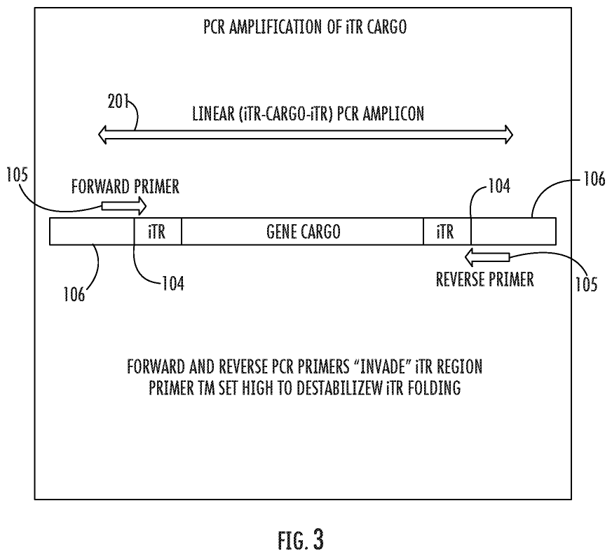

The PCR primers according to the subject invention are designed such that the 3′ terminus of both the forward and reverse primer pair only minimally invade the ITR sequences. As shown in FIG. 3, in exemplary embodiments, the 3′ termini of both the forward (105) and reverse (105) PCR primers are designed to invade and bind to only the last about 2-8 bases of the ITR A/A′ stem region (104), thereby inserting the flanking region bound PCR primers (105) into the A/A′ stem (104) over a region of only between 2-8 bases. This de minimis insertion into the A/A′ stem region (104) serves to destabilize the A/A′ stem region and, in turn, the overall structure of the ITR fold to facilitate successful high-fidelity PCR amplification of a [ITR-cargo-ITR] construct to create amplicons comprising the [ITR-cargo-ITR] (201). Experimentation has shown that design of primers that bind to more than approximately 10 bases of the ITR A/A′ stem leads to low amplification efficiencies, loss in accurate and/or the amplification of several side products.

In an alternative embodiment, the 3′ termini of both the forward and reverse PCR primer pair are designed to only bind to the last between 2 and 5 bases of the ITR A/A′ stem region (104).

In general, the [ITR-cargo-ITR] region is embedded in a larger DNA fragment and is thus flanked to either side by DNA flanking sequences (106), i.e. Flank-[ITR-cargo-ITR]-Flank. Having designed the 3′ termini of the PCR forward and reverse primers for minimal ITR insertion and binding as described above, the remainder of the PCR forward and reverse primers sequences are designed to bind to between 20-35 bases of the adjacent flanking sequences (106), thus yielding a PCR primer that is 30-40 bases in length and designed to span the junction between the ITR A/A′ stem (104) and flanking region DNA sequence (106). The 5′end of the PCR forward and reverse primers are kept long (20-35 bases) to allow for high affinity binding to the flanking region DNA sequence (to drive disruption of the stable ITR fold) and to ensure that forward and reverse primer binding is specific to the target template DNA sequence comprising the Flank-[ITR] junction.

Generally, PCR amplification reactions are performed as a series of three steps at the stated temperatures or within the stated temperature ranges: (i) denaturing step at 98° C.; (ii) annealing step at between 55° C. to 65° C.; and (iii) extension step at between 70° C. to 73° C.

In the present invention, the PCR amplification reaction is reduced to 2 steps, through the creation of a single high temperature annealing and extension step. In an embodiment, 2-step PCR amplification of a [ITR-cargo-ITR] construct is accomplished via the use of a denaturing step at 98° C. and a single combined high temperature annealing and extension step at above 70° C. In an exemplary embodiment, temperatures between 70° C. and 73° C. may be used. This results in high temperature annealing at above 70° C. versus the conventional range of 55° C. to 65° C. for an annealing step. The elimination of the lower temperature annealing in favor of high temperature annealing destabilizes the ITR structure by keeping temperature higher than 70° C. throughout the entire PCR amplification reaction. Without the use of an annealing temperature above 70° C. the secondary structure of the ITR sequence would form during the PCR reaction, thereby greatly diminishing amplification yield and/or fidelity.

Amplification of a [ITR-cargo-ITR] construct is further facilitated via the use of specific PCR enhancers. While the concept of PCR enhancers are well known in the art, including DMSO, PEG, glycerol, BSA, betaine and other osmolytes, the inventor has found that, while most osmolytes tested, such as DMSO, seem not to be effective in supporting PCR amplification of a [ITR-cargo-ITR] construct, the osmolyte betaine significantly increases PCR efficiency and fidelity specifically of a [ITR-cargo-ITR] construct when coupled with the other PCR modifications described herein. Betaine as a PCR enhancer is unique in that the inventor has shown betaine to stabilize DNA polymerases (including Taq Polymerase) against thermal denaturation, while selectively destabilizing the formation of G-C base pairs at elevated temperature due to selective solvation of free guanosine. Thus, the inventors have discovered that the unique polymerase stabilization and G-C base pair destabilization imparted by betaine are required to obtain adequate PCR yields from [ITR-cargo-ITR] constructs without significant side reactions. In an exemplary embodiment, betaine is used at 0.5M concentration in the PCR reaction. In other alternative embodiments, betaine is utilized at between 1M and 0.01M concentrations in the PCR reaction.

The PCR produced [ITR-cargo-ITR] construct may be transfected into packaging cell lines (such as HEK293 or other cell lines known in the art) along with conventional AAV helper and rep/cap plasmids to produce rAAV. The PCR produced [ITR-cargo-ITR] construct may also be transfected into packaging cell lines along with AAV helper and rep/cap constructs, wherein one or both constructs are amplicon polynucleotides manufactured by PCR. The packaging cell lines may be optimized for use with PCR produced [ITR-cargo-ITR] constructs and/or AAV helper and rep/cap constructs wherein one or both are manufactured by PCR. PCR-produced [ITR-cargo-ITR], AAV helper and rep/cap constructs may be produced by large-scale PCR. The large-scale PCR may be continuous flow.

Transfection into packaging cell lines may be accomplished via any methods known in the art. Exemplary methods include, without limitation, direct uptake, electroporation, chemical or other substance-based methods (e.g. calcium chloride, rubidium chloride, alcohol, DEAE-dextran, polyethylenimine (PEI)) lipofection, cationic liposomes, soluporation, lipid nanoparticles (LNP), cationic polymers, lipoplexes, synthetic branched dendrimers, microprojectile bombardment and cellular surgery. Viral transduction or transposon/transposase systems may also be used.

PCR-produced [iTR-cargo-iTR], AAV helper and/or rep/cap constructs may also be manufactured via methods and systems that mitigate PCR-based sequence error. Extremely high-fidelity polymerase such as Q5® polymerase (NEB Biolabs, Inc. USA) with an error rate less than 5.3.times.10⁻⁷ in the PCR reaction may be used. PCR conditions may also be optimized to increase fidelity. Large-scale PCR can be used in conjunction with high-fidelity polymerase to amplify [ITR-cargo-ITR], AAV helper and/or rep/cap constructs to economically create a high copy number of amplicons for use in rAAV manufacture.

After PCR amplification, the resultant [iTR-cargo-iTR], AAV helper and/or rep/cap construct amplicons may be sequence verified via NGS before transfection into packaging cell lines or a representative sample of the amplicons may be sequenced via NGS as part of quality control. In addition, post transfection, the packaging cell lines (or a representative sample thereof) may undergo RNA sequence analysis via NGS to ascertain whether the transfected cells are expressing the correct RNA sequence based on the desired sequence of the transfected amplicons. Post transfection, viral assembly and lysing of the packing cells, samples of the resultant rAAV may also be sequenced via NGS to confirm sequence accuracy. Samples of the resultant rAAV may also be interrogated via mass spectrometry to ensure correct structure and sequence. In addition, the cargo (transgene) sequence of the resultant rAAV may be specifically interrogated via NGS to ensure proper DNA sequence prior to introduction into a subject.

Production of rAAV Containing Single Polarity ssDNA Utilizing Forced Asymmetric PCR Primer Template Amplification to Produce Single Polarity [ITR-Cargo-ITR] Amplicon.

In an aspect of the invention, specialized [ITR-cargo-ITR] amplicons can also be used to produce single polarity rAAV vectors. While rAAV vectors containing exclusively positive (sense) polarity of ssDNA are set forth in this exemplary embodiment, the method and system disclosed herein can similarly by utilized to manufacture rAAV vectors containing only negative (antisense) polarity of ssDNA.

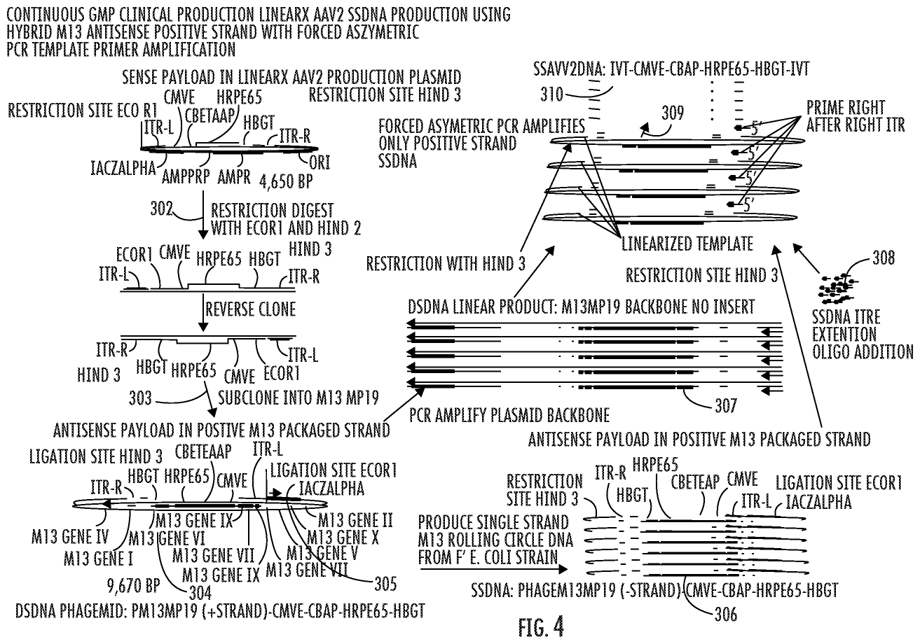

As shown in FIG. 4, the first step is preparation of the reverse (−) payload plasmid dsDNA PHAGEMID: pM13mp19 (+strand)-CMVe-CBAp-hRPE65-hBGt (301). The starting plasmid is a production plasmid containing the expression cassette for the transgene (cargo) of interest. This double-stranded dsDNA production plasmid, designated p-CMVe-CBAp-hRPE65-hBGt (301), may reside in a pUC18 plasmid or other commercially available cloning vector. This is a positive (sense) strand expression cassette plasmid where positive (sense) refers to the direction the transgene is transcribed from the DNA strand by mRNA from 5′ to 3′. Negative (antisense) refers to the reverse direction 3′ to 5′.

The cloning plasmid is digested with the restriction enzymes EcoR1 and Hind3 (302) to release and reverse the restricted expression cassette, which is purified and inserted into an M13mp19 plasmid precut with EcoR1 and Hind 3. As shown in FIG. 4, the expression cassette is subcloned in the reverse direction into the multiple cloning site of M13mp19 plasmid or other suitable cloning plasmid so that the negative strand of the expression cassette is placed into M13mp19 or other suitable cloning plasmid (303). After the expression cassette insert is subcloned into M13mp19 or other suitable cloning plasmid (303), it will provide the negative strand for a PCR amplification template. This will allow the negative strand to be used as a template once primed with the positive strand of the PCR amplification of the backbone using PCR primers amplifying the plasmid backbone. The antisense payload (305) in the positive M13mp19 packaged strand has a Hind3 site at the end of the expression vector for use in forced asymmetric PCR. This resulting plasmid is referred to as dsDNA PHAGEMID: pM13mp19 (+strand)-CMVe-CBAp-hRPE65-hBGt (304).

The second step as shown in FIG. 4 is using the dsDNA PHAGEMID: pM13mp19 (+strand)-CMVe-CBAp-hRPE65-hBGt (304) prepared in the first step to produce single stranded DNA (ssDNA) for two reactions. In the first reaction, the PHAGEMID: M13mp19 (+strand)-CMVe-CBAp-hRPE65-hBGt (304) is prepared for use as the ssDNA PCR template: phage M13mp19 (−strand)-CMVe-CBAp-hRPE65-hBGt (306) to receive the PCR long primer by infecting an E. coli with F. pilus to make the ssDNAphage genome containing the negative-stranded cassette insert with single strand M13 rolling circle DNA and making supernatants to purify the positive strand. In the second reaction, pM13mp19 (+strand)-CMVe-CBAp-hRPE65-hBGt (304) is used to PCR amplify the plasmid backbone to generate a dsDNA vector backbone without the insert, generating the dsDNA linear PCR product M13mp19 backbone no insert (307), which will allow a very long priming positive strand representing the plasmid backbone for the pM13mp19 (−strand)-CMVe-CBAp-hRPE65-hBGt dsDNA plasmid (306).

The two resulting products, M13mp19 backbone no insert (307) and the antisense payload in positive M13 packaged strand (306), along with addition of ssDNA ITR extension oligonucleotides (308), are all mixed, heated to denature, and annealed; the newly formed Hind3 site is cut to linearize the hybrid template. The resulting linearized moiety is then annealed to the primer, and the forced PCR reaction occurs (309); it expresses only the single strand positive transgene expression cassette with self-formed functional ITR ends form a [ITR-cargo-ITR] amplicon template. The [ITR-cargo-ITR] template using the reverse primer will allow forced asymmetric amplification of the [ITR-cargo-ITR] template, giving rise only to the positive strand [ITR-cargo-ITR] amplicons (310). When transfected, this specialized ssDNA [ITR-cargo-ITR] amplicon AAV template (310), denoted ssAVV2DNA: IVT-CMVe-CBAp-hRPE65-hBGt-IVT in FIG. 4, will give rise to only positive ssDNA strand containing rAAV. The vector may be transfected as described herein into modified HEK293 cells or other packaging cell lines.

In another embodiment, rAAV vectors containing a single polarity genome may be produced via modification to the one or both ITR sequences. Modifications may include one or more base deletions or insertions. The [ITR-cargo-ITR] amplicon, with one or more modifications to its ITR regions can then be amplified via PCR as disclosed herein and used to produce rAAV vectors according to the method and system set forth herein. Due to the modification of the ITR sequence, single polarity rAAV vectors can be produced. Modification of the ITR sequence may occur within the A, B, C, or D elements of one or both ITRs of the [ITR-cargo-ITR] construct, or any combination thereof. The wild-type A, B, C, and D element sequences of the ITR are shown in FIG. 1. In an embodiment, the ITR DNA sequence is modified within the D element.

Having now generally described the invention, the same will be more readily understood through reference to the following examples, which are provided by way of illustration, and are not intended to be limiting of the disclosed invention, unless specified.

### EXAMPLES

### Example 1—High Efficiency and High Fidelity PCR Amplifications from a Plasmid Containing an [ITR-Cargo-ITR] Construct

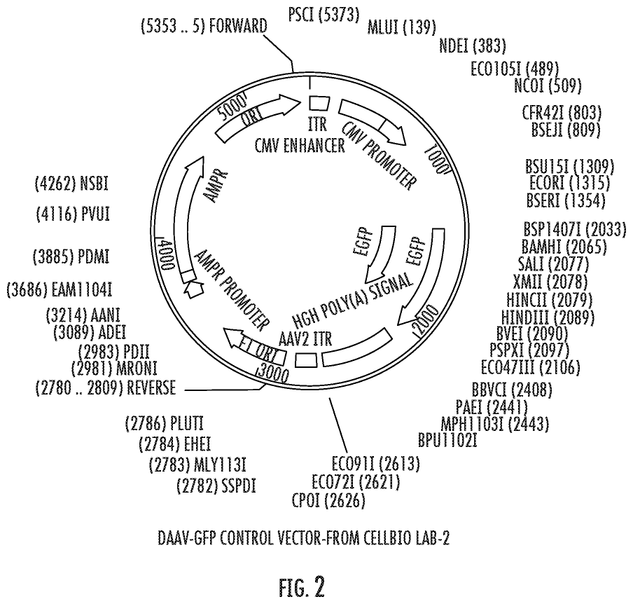

A commercially sourced GFP plasmid containing a cargo (transgene) expression cassette for EGFP, flanked by two ITR regions (part #: AAV-400, Cell Biolab Inc., San Diego, Calif.) (shown in FIG. 2) was used as a PCR template for a [ITR-cargo-ITR] construct (wherein the cargo is EGFP) and was subject to several PCR amplifications as follows:

Amplification #1

For PCR amplification #1, the following primer set was utilized:

Forward primer (AAV-GFP-F):
(SEQ ID NO: 1)
5′ CTTTTGCTGGCCTTTTGCTCACATGTCCTGC 3′
Reverse primer (AAV-GFP-R):
(SEQ ID NO: 2)
5′ GTAAGGAGAAAATACCGCATCAGGCGCCCC 3′

The PCR amplification reaction was carried out in 100 μL volume utilizing the following PCR reaction composition.

| | Final | | Volume |
|---|---|---|---|
| Composition | Concentration | | (μL) |
| PCR water | — | | 39.5 |
| Q5 5X buffer | 1X | | 20 |
| 5X GC enhancer | 1X | | 20 |
| dNTP 40 mM | 0.8 | mM | 2 |
| Q5 Polymerase 2 U/μl | 0.02 | U/μL | 1 |
| AAV-GFP-F | 0.5 | μM | 0.5 |
| AAV-GFP-R | 0.5 | μM | 0.5 |
| AAV-400-GFP plasmid | 1 ng/25 | μL | 4 |

| | | |
|---|---|---|
| 4M betaine | 0.5M | 12.5 |
| Total volume | | 100 |

The PCR reaction composition above was subjected to the below two-step PCR cycling parameters.

### PCR Cycling Parameters

| | Initial | | Annealing/ | Final |
|---|---|---|---|---|
| | Denature | Denature | Extension | Extension |
| | 98° C. | 98° C. | 72° C. | 72° C. |
| Duration | 30 sec. | 10 sec. | 3 min. | 2 sec. |

| | | | |
|---|---|---|---|
| Cycles | 1 | 28 | 1 |

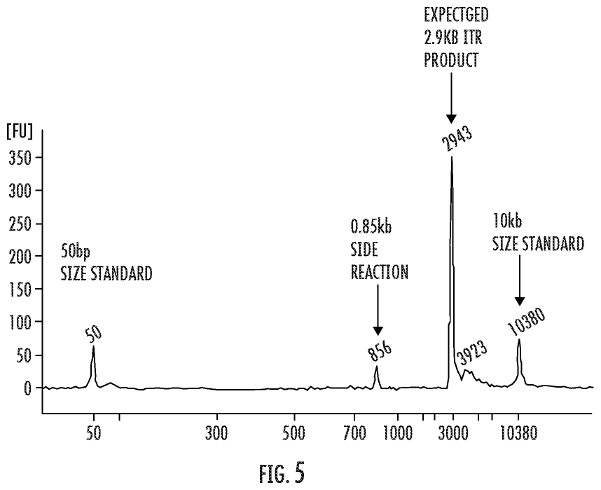

As shown in FIG. 5, the resultant [ITR-EGFP-ITR] amplicon produced by the above described PCR reaction was detected via electropherogram obtained via an Agilent Bioanalyzer. As shown below, a large amount of [ITR-EGFP-ITR] amplicon was detected with minimal side reactions.

Amplification #2

For amplification #2, the same commercially sourced plasmid (see. FIG. 2) containing the [ITR-EGFP-ITR] construct from amplification #1 was used again as a template for PCR amplification of the [ITR-EGFP-ITR] construct. The PCR cycling parameters were identical to amplification #1, but the inclusion of betaine as part of the PCR reaction composition was removed.

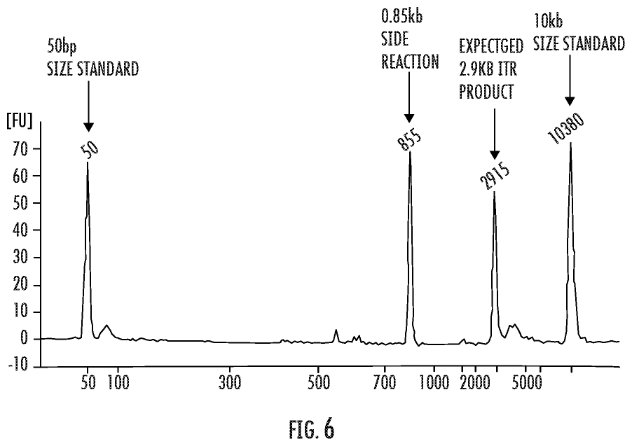

As can be seen in FIG. 6, an electropherogram obtained via an Agilent Bioanalyzer, the removal of betaine from the PCR reaction composition greatly reduced the yield of the target [ITR-EGFP-ITR] construct and significantly increased side reactions as compared to the amplicon produced by amplification #1.

Amplification #3

For amplification #3, the same commercially sourced plasmid (see. FIG. 2) containing the [ITR-EGFP-ITR] construct used in amplification #1 was again used as a PCR template. The PCR cycling parameters were identical to amplification #1, but the betaine in the PCR reaction composition was replaced with 5% DMSO (another osmolyte).

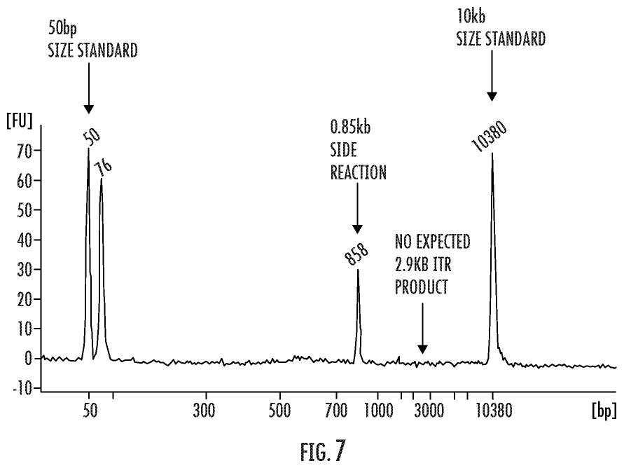

As can be seen in FIG. 7, an electropherogram obtained via an Agilent Bioanalyzer, the substitution of 5% DMSO for betaine in the PCR reaction composition resulted in the failure of [ITR-EGFP-ITR] construct to amplify and also resulted in several undesirable side reactions.

Amplification #4

A fourth PCR amplification was conducted, again using the same commercially sourced plasmid used in amplification #1 as the [ITR-EGFP-ITR] PCR template. The forward and reverse primers used were as follows:

Forward primer:
(SEQ ID NO: 3)
5′ TCCTGGCCTTTTGCTGGCCTTTTGCTCACATGTCCTG3′
Reverse primer:
(SEQ ID NO: 2)
5′ GTAAGGAGAAAATACCGCATCAGGCGCCCC3′

The PCR reaction composition was identical to amplification #1, but 0.75M betaine was used versus the 0.5M betaine used in amplification #1. In addition, the PCR cycling parameters from amplification #1 were adjusted to include a 5 minute annealing/extension time at 72° C.

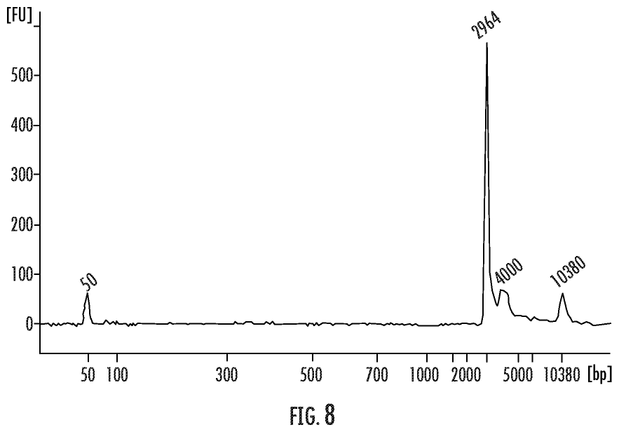

As seen in FIG. 8, another electropherogram obtained via an Agilent Bioanalyzer, these modifications resulted in increased yield of the target [ITR-EGFP-ITR] construct and further reduced undesirable side reactions, resulting in a high-yield high-fidelity [ITR-EGFP-ITR] amplicon.

### Pharmaceutical Compositions

In another aspect, pharmaceutical compositions are provided. The pharmaceutical composition comprises a recombinant adeno-associated virus (rAAV) produced using the synthetic process as described herein and, optionally, a pharmaceutically acceptable carrier or diluent. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

An rAAV, produced using the synthetic process as described herein can be incorporated into pharmaceutical compositions suitable for administration to a subject for in vivo delivery to cells, tissues, or organs of the subject. Typically, the pharmaceutical composition comprises an rAAV as disclosed herein and a pharmaceutically acceptable carrier. For example, an rAAV produced using the synthetic process as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, or liposomes. Sterile injectable solutions can be prepared by incorporating the synthetically produced rAAV compound in the required amount in an appropriate buffer with one or a combination of ingredients enumerated above, as required, followed by filtered sterilization to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising an rAAV, produced using the synthetic process as described herein, can be formulated to deliver a transgene for various purposes to the cell, e.g., cells of a subject.

An rAAV produced using the synthetic process as described herein as disclosed herein can be incorporated into a pharmaceutical composition suitable for systemic, intra-amniotic, intrathecal, intracranial, intra-arterial, intravenous, intralymphatic, intraperitoneal, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more rAAV produced using the synthetic process as described herein to a host cell. Methods of delivery can include lipofection, nucleofection, microinjection, biolistics, liposomes, immunoliposomes, polycation or lipid:nucleic acid conjugates, and lipofection reagents are sold commercially. Delivery can be to cells (e.g., in vitro or ex vivo administration) or target tissues (e.g., in vivo administration).

An rAAV produced using the synthetic process as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering are well known to those of skill in the art, and, although more than one route can be used to administer a particular composition, a particular route can often provide a more immediate and more effective reaction than another route.

### Methods of Treatment

The technology described herein also demonstrates methods for making, as well as methods of using the disclosed synthetically produced rAAV in a variety of ways, including, for example, ex situ, in vitro and in vivo applications, methodologies, diagnostic procedures, and/or gene therapy regimens

Provided herein is a method of treating a disease or disorder in a subject comprising introducing into a target cell in need thereof (for example, a muscle cell or tissue, or other affected cell type) of the subject a therapeutically effective amount of a synthetically produced rAAV, optionally with a pharmaceutically acceptable carrier. The synthetically produced rAAV implemented comprises a nucleotide sequence of interest useful for treating the disease. In particular, the synthetically produced rAAV may comprise a desired exogenous DNA sequence operably linked to control elements capable of directing transcription of the desired polypeptide, protein, or oligonucleotide encoded by the exogenous DNA sequence when introduced into the subject. The synthetically produced rAAV can be administered via any suitable route as provided above, and elsewhere herein.

Disclosed herein are an rAAV compositions and formulations that include one or more of the synthetically produced rAAV of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction, typically, in a human.

Another aspect of the technology described herein provides a method for providing a subject in need thereof with a diagnostically- or therapeutically-effective amount of a synthetically produced rAAV, the method comprising providing to a cell, tissue or organ of a subject in need thereof, an amount of the synthetically produced rAAV as disclosed herein; and for a time effective to enable expression of the transgene from the rAAV thereby providing the subject with a diagnostically- or a therapeutically-effective amount of the protein, peptide, nucleic acid expressed by an rAAV. In one embodiment, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed synthetically produced rAAV, in an amount and for a time sufficient to diagnose, prevent, treat or ameliorate the one or more symptoms of the disease, disorder, dysfunction, injury, abnormal condition, or trauma in the subject. In one embodiment, the subject is human.

Another aspect is use of the synthetically produced rAAV as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, synthetically produced rAAV can be used to deliver transgenes to bring a normal gene into affected tissues for replacement therapy, as well, in some embodiments, to create animal models for the disease using antisense mutations. For unbalanced disease states, synthetically produced rAAV can be used to create a disease state in a model system, which could then be used in efforts to counteract the disease state. Thus the synthetically produced rAAV and methods disclosed herein permit the treatment of genetic diseases. As used herein, a disease state is treated by partially or wholly remedying the deficiency or imbalance that causes the disease or makes it more severe.

### Host Cells:

In some embodiments, the synthetically produced rAAV delivers the transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including synthetically produced rAAV as described herein. Thus, one can use multiple host cells depending on the purpose as is obvious to the skilled artisan. A construct or synthetically produced rAAV including donor sequence is introduced into a host cell so that the donor sequence is maintained as a chromosomal integrant. The term host cell encompasses any progeny of a parent cell that is not identical to the parent cell due to mutations that occur during replication. The choice of a host cell will to a large extent depend upon the donor sequence and its source. The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered the synthetically produced rAAV ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

Exemplary Transgenes and Diseases to be Treated with an rAAV

An rAAV produced using the synthetic process as described herein are also useful for correcting a defective gene. A synthetically produced rAAV or a composition thereof can be used in the treatment of any hereditary disease. As a non-limiting example, the synthetically produced rAAV or a composition thereof can treat diseases of the liver, brain, heart, muscle, eyes, lungs, kidneys and intestines, e.g. can be used in the treatment of transthyretin amyloidosis (ATTR), an orphan disease where the mutant protein misfolds and aggregates in nerves, the heart, the gastrointestinal system etc. It is contemplated herein that the disease can be treated by deletion of the mutant disease gene (mutTTR) using the synthetically produced rAAV described herein. Such treatments of hereditary diseases can halt disease progression and may enable regression of an established disease or reduction of at least one symptom of the disease by at least 10%.

In another embodiment, a synthetically produced an rAAV or a composition thereof can be used in the treatment of ornithine transcarbamylase deficiency (OTC deficiency), hyperammonemia or other urea cycle disorders, which impair a neonate or infant's ability to detoxify ammonia. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom OTC and/or an improvement in the quality of life for a subject having OTC deficiency. In one embodiment, a nucleic acid encoding OTC can be inserted behind the albumin endogenous promoter for in vivo protein replacement.

In another embodiment, a synthetically produced rAAV can be used in the treatment of phenylketonuria (PKU) by delivering a nucleic acid sequence encoding a phenylalanine hydroxylase enzyme to reduce buildup of dietary phenylalanine, which can be toxic to PKU sufferers. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of PKU and/or an improvement in the quality of life for a subject having PKU. In one embodiment, a nucleic acid encoding phenylalanine hydroxylase can be inserted behind the albumin endogenous promoter for in vivo protein replacement.

In another embodiment, a synthetically produced rAAV can be used in the treatment of glycogen storage disease (GSD) by delivering a nucleic acid sequence encoding an enzyme to correct aberrant glycogen synthesis or breakdown in subjects having GSD. Non-limiting examples of enzymes that can be delivered and expressed using the synthetically produced an rAAV and methods as described herein include glycogen synthase, glucose-6-phosphatase, acid-alpha glucosidase, glycogen debranching enzyme, glycogen branching enzyme, muscle glycogen phosphorylase, liver glycogen phosphorylase, muscle phosphofructokinase, phosphorylase kinase, glucose transporter-2 (GLUT-2), aldolase A, β-enolase, phosphoglucomutase-1 (PGM-1), and glycogenin-1. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of GSD and/or an improvement in the quality of life for a subject having GSD. In one embodiment, a nucleic acid encoding an enzyme to correct aberrant glycogen storage can be inserted behind the albumin endogenous promoter for in vivo protein replacement.

The synthetically produced rAAV described herein are also contemplated for use in the treatment of any of Leber congenital amaurosis (LCA), polyglutamine diseases, including polyQ repeats, and α-1 antitrypsin deficiency (A1AT). LCA is a rare congenital eye disease resulting in blindness, which can be caused by a mutation in any one of the following genes: GUCY2D, RPE65, SPATA7, AIPL1, LCA5, RPGRIP1, CRX, CRB1, NMNAT1, CEP290, IMPDH1, RD3, RDH12, LRAT, TULP1, KCNJ13, GDF6 and/or PRPH2. It is contemplated herein that the rAAV and compositions and methods as described herein can be adapted for delivery of one or more of the genes associated with LCA in order to correct an error in the gene(s) responsible for the symptoms of LCA. Polyglutamine diseases include, but are not limited to: dentatorubropallidoluysian atrophy, Huntington's disease, spinal and bulbar muscular atrophy, and spinocerebellar ataxia types 1, 2, 3 (also known as Machado-Joseph disease), 6, 7, and 17. A1AT deficiency is a genetic disorder that causes defective production of alpha-1 antitrypsin, leading to decreased activity of the enzyme in the blood and lungs, which in turn can lead to emphysema or chronic obstructive pulmonary disease in affected subjects. Treatment of a subject with an A1AT deficiency is specifically contemplated herein using an rAAV or compositions thereof as outlined herein. It is contemplated herein that an rAAV comprising a nucleic acid encoding a desired protein for the treatment of LCA, polyglutamine diseases or A1AT deficiency can be administered to a subject in need of treatment.

In further embodiments, the compositions comprising a synthetically produced rAAV as described herein can be used to deliver a viral sequence, a pathogen sequence, a chromosomal sequence, a translocation junction (e.g., a translocation associated with cancer), a non-coding RNA gene or RNA sequence, a disease associated gene, among others.

Any nucleic acid or target gene of interest may be delivered or expressed by a synthetically produced rAAV as disclosed herein. Target nucleic acids and target genes include, but are not limited to nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the target nucleic acids or target genes that are targeted by the synthetically produced rAAV as described herein encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, a gene target or transgene for expression by the synthetically produced rAAV as disclosed herein can encode, for example, but is not limited to, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder, for example, in a human.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of an rAAV described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein.

As noted herein, a synthetically produced rAAV as disclosed herein can encode a protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

The synthetically produced rAAV are also useful for ablating gene expression. For example, in one embodiment an rAAV can be used to express an antisense nucleic acid to induce knockdown of a target gene. In some embodiments, a synthetically produced rAAV is useful for correcting a defective gene by expressing a transgene that targets the diseased gene.

In alternative embodiments, the synthetically produced rAAV are used for insertion of an expression cassette for expression of a therapeutic protein or reporter protein in a safe harbor gene, e.g., in an inactive intron. In certain embodiments, a promoter-less cassette is inserted into the safe harbor gene. In such embodiments, a promoter-less cassette can take advantage of the safe harbor gene regulatory elements (promoters, enhancers, and signaling peptides).

In some embodiments, the synthetically produced rAAV are used for expressing a transgene, or knocking out or decreasing expression of a target gene in a T cell, e.g., to engineer the T cell for improved adoptive cell transfer and/or CAR-T therapies. In some embodiments, the rAAV vector as described herein can express transgenes that knock-out genes.

### Single Gene Disorders

In general, the rAAV vector produced by the synthetic methods as disclosed herein can be used to deliver any transgene in accordance with the description above to treat, prevent, or ameliorate the symptoms associated with any disorder related to gene expression. In particular, the methods of the invention can be used to treat/prevent/ameliorate the symptoms of single gene disorders. Single gene disorders are caused by DNA changes in one particular gene, and often have predictable inheritance patterns. Such disorders include, for example, Cystic Fibrosis, Galactosemia, Huntington Disease, Sickle Cell Anemia, Adenosine deaminase (ADA) deficiency, Fragile X Syndrome, Spinal Muscular Dystrophy, alpha-1-antitrypsin deficiency, Marfan syndrome, neurofibromatosis, retinoblastoma, polydactyly, phenylketonuria, Tay-Sachs disease, hemophilia A, muscular dystrophies (e.g., Duchenne, Becker), and glucose-6-phosphate dehydrogenase deficiency, and Rett syndrome.

### Additional Diseases for Gene Therapy

In general, the rAAV vector produced by the synthetic methods as disclosed herein can be used to deliver any transgene in accordance with the description above to treat, prevent, or ameliorate the symptoms associated with any disorder related to gene expression. Illustrative disease states include, but are not-limited to: diseases of the lung, hemophilia A, hemophilia B, thalassemia, anemia and other blood disorders, AIDS, Alzheimer's disease, Parkinson's disease, amyotrophic lateral sclerosis, epilepsy, and other neurological disorders, cancer, diabetes mellitus, Hurler's disease, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, an rAAV produced by the synthetic production methods as described herein can be advantageously used in the treatment of individuals with metabolic disorders (e.g., ornithine transcarbamylase deficiency).

In some embodiments, an rAAV produced by the synthetic production methods as described herein can be used to treat, ameliorate, and/or prevent a disease or disorder caused by mutation in a gene or gene product. Exemplary diseases or disorders that can be treated with an rAAV include, but are not limited to, metabolic diseases or disorders (e.g., Fabry disease, Gaucher disease, glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., thalassemia, and anemia); cancers and tumors, and genetic diseases or disorders.

As still a further aspect an rAAV produced by the synthetic production methods as described herein may be employed to deliver a heterologous nucleotide sequence in situations in which it is desirable to regulate the level of transgene expression (e.g., transgenes encoding hormones or growth factors, as described herein).

Accordingly, in some embodiments, an rAAV produced by the synthetic production methods as described herein can be used to correct an abnormal level and/or function of a gene product (e.g., an absence of, or a defect in, a protein) that results in the disease or disorder. The rAAV can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MLD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In alternative embodiments, an rAAV produced by the synthetic production methods as described herein can be used to provide an antisense nucleic acid to a cell in vitro or in vivo.

In some embodiments, exemplary transgenes encoded by an rAAV produced by the synthetic production methods as described herein, include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and -β, and the like), receptors (e.g., tumor necrosis factor receptor). In some exemplary embodiments, the transgene encodes a monoclonal antibody specific for one or more desired targets. In some exemplary embodiments, more than one transgene is encoded by an rAAV. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by an rAAV produced by the synthetic production methods as described herein can be used for the treatment of muscular dystrophy in a subject in need thereof, the method comprising: administering a treatment-, amelioration-, or prevention-effective amount of an rAAV described herein, wherein the rAAV comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the synthetically produced an rAAV can be administered to skeletal, diaphragm and/or cardiac muscle as described elsewhere herein.

In some embodiments, an rAAV produced by the synthetic production methods as described herein can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid a glucosidase]). Other suitable proteins for treating, ameliorating, and/or preventing metabolic disorders are described above.

In other embodiments, an rAAV produced by the synthetic production methods as described herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the invention relates to a method of treating, ameliorating, and/or preventing congenital heart failure or PAD in a subject in need thereof, the method comprising administering an rAAV produced by the synthetic production methods as described herein to a mammalian subject, wherein the rAAV comprises a transgene encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (β-ARKct), inhibitor 1 of protein phosphatase 1, S100A1, parvalbumin, adenylyl cyclase type 6, a molecule that effects G-protein coupled receptor kinase type 2 knockdown such as a truncated constitutively active β-ARKct, Pim-1, PGC-1α, SOD-1, SOD-2, EC-SOD, kallikrein, HIF, thymosin-β4, mir-1, mir-133, mir-206 and/or mir-208.

In some embodiments, an rAAV produced by the synthetic production methods as described herein can be administered to the lungs of a subject by any suitable means, optionally by administering an aerosol suspension of respirable particles comprising the rAAV, which the subject inhales. The respirable particles can be liquid or solid. Aerosols of liquid particles comprising the rAAV may be produced by any suitable means, such as with a pressure-driven aerosol nebulizer or an ultrasonic nebulizer, as is known to those of skill in the art. Aerosols of solid particles comprising an rAAV produced by the synthetic production methods as described herein may likewise be produced with any solid particulate medicament aerosol generator, by techniques known in the pharmaceutical art.

In some embodiments, an rAAV produced by the synthetic production methods as described herein can be administered to tissues of the CNS (e.g., brain, eye). In particular embodiments, an rAAV produced by the synthetic production methods as described herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Ocular disorders that may be treated, ameliorated, or prevented with an rAAV produced by the synthetic production methods as described herein include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, an rAAV produced by the synthetic production methods as described herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with an rAAV of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by an rAAV produced by the synthetic production methods as described herein. One or more anti-inflammatory factors can be expressed by intraocular (e.g., vitreous or anterior chamber) administration of an rAAV produced by the synthetic production methods as described herein. In other embodiments, ocular diseases or disorders characterized by retinal degeneration (e.g., retinitis pigmentosa) can be treated, ameliorated, or prevented by the rAAV of the invention. Intraocular (e.g., vitreal administration) of an rAAV produced by the synthetic production methods as described herein encoding one or more neurotrophic factors can be used to treat such retinal degeneration-based diseases. In some embodiments, diseases or disorders that involve both angiogenesis and retinal degeneration (e.g., age-related macular degeneration) can be treated with an rAAV produced by the synthetic production methods as described herein. Age-related macular degeneration can be treated by administering an rAAV produced by the synthetic production methods as described herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the rAAV as disclosed herein. Accordingly, such agents include N-methyl-D-aspartate (NMDA) antagonists, cytokines, and neurotrophic factors, can be delivered intraocularly, optionally intravitreally using an rAAV produced by the synthetic production methods as described herein.

In other embodiments, an rAAV produced by the synthetic production methods as described herein may be used to treat seizures, e.g., to reduce the onset, incidence or severity of seizures. The efficacy of a therapeutic treatment for seizures can be assessed by behavioral (e.g., shaking, tics of the eye or mouth) and/or electrographic means (most seizures have signature electrographic abnormalities). Thus, an rAAV produced by the synthetic production methods as described herein can also be used to treat epilepsy, which is marked by multiple seizures over time. In one representative embodiment, somatostatin (or an active fragment thereof) is administered to the brain using an rAAV produced by the synthetic production methods as described herein to treat a pituitary tumor. According to this embodiment, an rAAV produced by the synthetic production methods as described herein encoding somatostatin (or an active fragment thereof) is administered by microinfusion into the pituitary. Likewise, such treatment can be used to treat acromegaly (abnormal growth hormone secretion from the pituitary).

In other embodiments, an rAAV produced by the synthetic production methods as described herein may be used to treat neuromuscular diseases, and familial lipoprotein lipase deficiency. The rAAV is also the best choice for the transduction of slowly dividing cells such as myocytes or cardiomyocytes.

Another aspect of the invention relates to the use of an rAAV produced by the synthetic production methods as described herein to produce antisense RNA, RNAi or other functional RNA (e.g., a ribozyme) for systemic delivery to a subject in vivo. Accordingly, in some embodiments, an rAAV produced by the synthetic production methods as described herein can comprise a transgene that encodes an antisense nucleic acid, a ribozyme, RNAs that affect spliceosome-mediated trans-splicing, interfering RNAs (RNAi) that mediate gene silencing (see, Sharp et al., or other non-translated RNAs, such as “guide” RNAs, and the like.

In some embodiments, an rAAV produced by the synthetic production methods as described herein can further also comprise a transgene that encodes a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, synthetically produced rAAV comprising a transgene encoding a reporter polypeptide may be used for diagnostic purposes or as markers of the rAAV activity in the subject to which they are administered.

In some embodiments, an rAAV produced by the synthetic production methods as described herein can comprise a transgene or a heterologous nucleotide sequence that shares homology with, and recombines with a locus on the host chromosome. This approach may be utilized to correct a genetic defect in the host cell.

In some embodiments, an rAAV produced by the synthetic production methods as described herein can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, Coronavirus (e.g., CoViD-19 spike protein and its variants), and the like.

### Administration

In particular embodiments, more than one administration (e.g., two, three, four or more administrations) may be employed to achieve the desired level of gene expression over a period of various intervals, e.g., daily, weekly, monthly, yearly, etc.

Exemplary modes of administration of an rAAV produced using the synthetic process as described herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of an rAAV produced using the synthetic process as described herein can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the synthetically produced rAAV can also be to a tumor (e.g., in or near a tumor or a lymph node). The most suitable route in any given case will depend on the nature and severity of the condition being treated, ameliorated, and/or prevented and on the nature of the particular rAAV that is being used. Additionally, an rAAV produced using the synthetic process as described herein permits one to administer more than one transgene in a single rAAV, or multiple rAAV (e.g. an rAAV cocktail).

Administration of an rAAV produced using the synthetic process as described herein to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The synthetically produced rAAV can be delivered to skeletal muscle by intravenous administration, intra-arterial administration, intraperitoneal administration, limb perfusion, and/or direct intramuscular injection. In particular embodiments, the rAAV as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In certain embodiments, an rAAV produced using the synthetic process as described herein can be administered without employing “hydrodynamic” techniques.

Administration of an rAAV produced using the synthetic process as described herein to cardiac muscle includes administration to the left atrium, right atrium, left ventricle, right ventricle and/or septum. The synthetically produced an rAAV as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, an rAAV produced using the synthetic process as described herein is administered to skeletal muscle, diaphragm muscle and/or cardiac muscle (e.g., to treat, ameliorate and/or prevent muscular dystrophy or heart disease (e.g., PAD or congestive heart failure).

### Ex Vivo Treatment

In some embodiments, cells are removed from a subject, an rAAV produced using the synthetic process as described herein is introduced therein, and the cells are then replaced back into the subject. Methods of removing cells from subject for treatment ex vivo, followed by introduction back into the subject are known in the art. Alternatively, an rAAV produced using the synthetic process as described herein is introduced into cells from another subject, into cultured cells, or into cells from any other suitable source, and the cells are administered to a subject in need thereof.

Cells transduced with an rAAV produced using the synthetic process as described herein are preferably administered to the subject in a “therapeutically-effective amount” in combination with a pharmaceutical carrier. Those of ordinary skill in the art will appreciate that the therapeutic effects need not be complete or curative, as long as some benefit is provided to the subject.

In some embodiments, an rAAV produced using the synthetic process as described herein can encode a transgene (sometimes called a heterologous nucleotide sequence) that is any polypeptide that is desirably produced in a cell in vitro, ex vivo, or in vivo. For example, in contrast to the use of the rAAV in a method of treatment as previously discussed herein, in some embodiments an rAAV produced using the synthetic process as described herein may be introduced into cultured cells and the expressed gene product isolated therefrom, e.g., for the production of antigens or vaccines.

An rAAV produced using the synthetic process as described herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, an rAAV produced using the synthetic process as described herein may be introduced into the cell using the methods as disclosed herein, as well as other methods known in the art. An rAAV produced using the synthetic process as described herein disclosed herein are preferably administered to the cell in a biologically-effective amount. If an rAAV produced using the synthetic process as described herein is administered to a cell in vivo (e.g., to a subject), a biologically-effective amount of an rAAV is an amount that is sufficient to result in transduction and expression of the transgene in a target cell.

### Dose Ranges

In vivo and/or in vitro assays can optionally be employed to help identify optimal dosage ranges for use of the synthetically produced an rAAV. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

An rAAV produced using the synthetic process as described herein is administered in sufficient amounts to transfect the cells of a desired tissue and to provide sufficient levels of gene transfer and expression without undue adverse effects. Conventional and pharmaceutically acceptable routes of administration include direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a synthetically produced rAAV required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a synthetically produced rAAV dose range to treat a patient having a particular disease or disorder based on the aforementioned factors, as well as other factors that are well known in the art.

Dosage regime can be adjusted to provide the optimum therapeutic response. For example, an rAAV can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether an rAAV are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to about 100 g of an rAAV. If exosomes or microparticles are used to deliver an rAAV produced using the synthetic process as described herein, then a therapeutically effective dose can be determined experimentally, but is expected to deliver from about 1 μg to about 100 g of vector. Moreover, a therapeutically effective dose is an amount an rAAV that expresses a sufficient amount of the transgene to have an effect on the subject that results in a reduction in one or more symptoms of the disease, but does not result in significant off-target or significant adverse side effects.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of an rAAV produced using the synthetic process as described herein to be delivered to cells (1×10⁶ cells) will be on the order of about 0.1 to 100 μg an rAAV, preferably 1 to 20 μg, and more preferably about 1 to 15 μg or 8 to 10 μg.

Treatment can involve administration of a single dose or multiple doses. In some embodiments, more than one dose can be administered to a subject; in fact multiple doses can be administered as needed.

In some embodiments, a dose of a synthetically produced rAAV is administered to a subject no more than once per calendar day (e.g., a 24-hour period). In some embodiments, a dose of a synthetically produced rAAV is administered to a subject no more than once per 2, 3, 4, 5, 6, or 7 calendar days. In some embodiments, a dose of a synthetically produced rAAV is administered to a subject no more than once per calendar week (e.g., 7 calendar days). In some embodiments, a dose of a synthetically produced rAAV is administered to a subject no more than bi-weekly (e.g., once in a two calendar week period). In some embodiments, a dose of a synthetically produced rAAV is administered to a subject no more than once per calendar month (e.g., once in 30 calendar days). In some embodiments, a dose of a synthetically produced rAAV is administered to a subject no more than once per six calendar months. In some embodiments, a dose of a synthetically produced rAAV is administered to a subject no more than once per calendar year (e.g., 365 days or 366 days in a leap year).

### Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### Various Applications

The rAAV produced using the synthetic process as described herein can be used to deliver a transgene for various purposes as described above. In some embodiments, a transgene can encode a protein or be a functional RNA, and in some embodiments, can be a protein or functional RNA that is modified for research purposes, e.g., to create a somatic transgenic animal model harboring one or more mutations or a corrected gene sequence, e.g., to study the function of the target gene. In another example, the transgene encodes a protein or functional RNA to create an animal model of disease.

In some embodiments, the transgene encodes one or more peptides, polypeptides, or proteins, which are useful for the treatment, amelioration, or prevention of disease states in a mammalian subject. The transgene expressed by the synthetically produced rAAV is administered to a patient in a sufficient amount to treat a disease associated with an abnormal gene sequence, which can result in any one or more of the following: reduced expression, lack of expression or dysfunction of the target gene.

In some embodiments, an rAAV produced using the synthetic process as described herein are envisioned for use in diagnostic and screening methods, whereby a transgene is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the synthetically produced an rAAV disclosed herein.

Additionally, the present invention provides compositions, as well as therapeutic and/or diagnostic kits that include one or more of the disclosed rAAV, produced using the synthetic process as described herein, formulated with one or more additional ingredients, or prepared with one or more instructions for their use.

A cell to be administered an rAAV produced using the synthetic process as described herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

Although the invention has been described with reference to the above examples and embodiments, it is not intended that such references be constructed as limitations upon the scope of this invention except as set forth in the following claims.

1. A pharmaceutical composition comprising the recombinant adeno-associated virus (rAAV) and optionally, an excipient, wherein the rAAV is produced by a method comprising:
obtaining a desired template DNA sequence containing a [ITR-cargo-ITR] DNA sequence motif;
designing a PCR primer pair such that the 3′ terminus of both the forward and reverse PCR primers overlap only about the last 2-8 bases of the A/A′ ITR sequences and the 5′ terminus of both the forward and reverse PCR primers extend into about 20-35 bases of the flanking sequences;
performing a PCR amplification reaction with PCR cycling parameters comprising a combined annealing/extension step at a temperature greater than 70° C., wherein the PCR amplification reaction contains one or more osmolytes, thereby producing a plurality of amplicon polynucleotides containing the desired [ITR-cargo-ITR] DNA sequence motif;
obtaining a quantity of the AAV rep/cap DNA sequence;
obtaining a quantity of AAV helper DNA sequence;
transfecting the amplicon polynucleotides containing the desired [ITR-cargo-ITR] DNA sequence motif, the AAV rep/cap DNA sequence and the AAV helper DNA sequence into a packaging cell line;
expanding the packaging cell line;
lysing cells of the packaging cell line;
and purifying the lysed cells to collect a quantity of rAAV.

2. The pharmaceutical composition of claim 1, wherein the AAV rep/cap DNA sequence is contained in a DNA plasmid.

3. The pharmaceutical composition of claim 1, wherein the AAV rep/cap DNA sequence is an amplicon polynucleotide.

4. The pharmaceutical composition of claim 1, wherein the AAV helper DNA sequence is an amplicon polynucleotide.

5. The pharmaceutical composition of claim 1, wherein the AAV helper DNA sequence is contained in plasmid DNA.

6. The pharmaceutical composition of claim 1, wherein both the AAV helper and rep/cap DNA sequences are amplicon polynucleotides.

7. The pharmaceutical composition of claim 1, wherein both the AAV helper and rep/cap DNA sequences are contained in DNA plasmids.

8. The pharmaceutical composition of claim 1, wherein the osmolyte is betaine.

9. A method for delivering a therapeutic protein to a subject, the method comprising: administering to a subject a composition comprising the recombinant adeno-associated virus (rAAV), wherein the rAAV is produced by a method comprising:
obtaining a desired template DNA sequence containing a [ITR-cargo-ITR] DNA sequence motif;
designing a PCR primer pair such that the 3′ terminus of both the forward and reverse PCR primers overlap only about the last 2-8 bases of the A/A′ ITR sequences and the 5′ terminus of both the forward and reverse PCR primers extend into about 20-35 bases of the flanking sequences;
performing a PCR amplification reaction with PCR cycling parameters comprising a combined annealing/extension step at a temperature greater than 70° C., wherein the PCR amplification reaction contains one or more osmolytes, thereby producing a plurality of amplicon polynucleotides containing the desired [ITR-cargo-ITR] DNA sequence motif;
obtaining a quantity of the AAV rep/cap DNA sequence;
obtaining a quantity of AAV helper DNA sequence;
transfecting the amplicon polynucleotides containing the desired [ITR-cargo-ITR] DNA sequence motif, the AAV rep/cap DNA sequence and the AAV helper DNA sequence into a packaging cell line;
expanding the packaging cell line;
lysing cells of the packaging cell line;
and purifying the lysed cells to collect a quantity of rAAV,
wherein at least one heterologous nucleotide sequence encodes a therapeutic protein.

10. The method of claim 9, wherein the therapeutic protein is an immunogen.

11. The method of claim 10, wherein the immunogen is from human immunodeficiency virus, influenza virus, gag proteins, tumor antigens, cancer antigens, bacterial antigens, viral antigens, Coronavirus, or CoViD-19.

12. The method of claim 11, wherein the therapeutic protein is a spike protein.

13. The method of claim 9, wherein the therapeutic protein is delivered to a neural cell, lung cell, retinal cell, epithelial cell, muscle cell, pancreatic cell, hepatic cell, myocardial cell, bone cell, hematopoietic stem cell, spleen cell, keratinocyte, fibroblast, endothelial cell, prostate cell, and/or germ cell.

14. A method of treating a disease in a subject in need, the method comprising administering to the subject a composition comprising the recombinant adeno-associated virus (rAAV), wherein the rAAV is produced by a method comprising:
obtaining a desired template DNA sequence containing a [ITR-cargo-ITR] DNA sequence motif;
designing a PCR primer pair such that the 3′ terminus of both the forward and reverse PCR primers overlap only about the last 2-8 bases of the A/A′ ITR sequences and the 5′ terminus of both the forward and reverse PCR primers extend into about 20-35 bases of the flanking sequences;
performing a PCR amplification reaction with PCR cycling parameters comprising a combined annealing/extension step at a temperature greater than 70° C., wherein the PCR amplification reaction contains one or more osmolytes, thereby producing a plurality of amplicon polynucleotides containing the desired [ITR-cargo-ITR] DNA sequence motif;
obtaining a quantity of the AAV rep/cap DNA sequence;
obtaining a quantity of AAV helper DNA sequence;
transfecting the amplicon polynucleotides containing the desired [ITR-cargo-ITR] DNA sequence motif, the AAV rep/cap DNA sequence and the AAV helper DNA sequence into a packaging cell line;
expanding the packaging cell line;
lysing cells of the packaging cell line;
and purifying the lysed cells to collect a quantity of rAAV,
wherein at least one heterologous nucleotide sequence encodes a therapeutic protein,
wherein the subject is treated.

15. The method of claim 14, wherein the disease is human immunodeficiency virus, influenza virus, cancer, Coronavirus or CoViD-19.

16. The method of claim 14 wherein the disease is a lung disease, a blood disorder, AIDS, a neurological disorder, cancer, diabetes mellitus, a muscular dystrophy, Hurler's disease, a metabolic defect, an ocular disease, a mitochondriopathy, a myopathy, a liver disease, a kidney disease or a heart disease.

17. The method of claim 16 wherein the disease is hemophilia A, hemophilia B, thalassemia, anemia, Alzheimer's disease, Parkinson's disease, amyotrophic lateral sclerosis, epilepsy, a retinal degenerative disease, Leber's hereditary optic neuropathy, Leigh syndrome, subacute sclerosing encephalopathy, facioscapulohumeral myopathy or cardiomyopathy.

18. The method of claim 16 wherein the disease is Fabry disease, Gaucher disease, glycogen storage disease, ornithine transcarbamylase deficiency, metachromatic leukodystrophy, mucopolysaccharidosis Type II or progressive familial intrahepatic cholestasis.

19. The method of claim 14 wherein the disease is caused by a single gene disorder.

20. The method of claim 19 wherein the single gene disorder is cystic fibrosis, galactosemia, Huntington Disease, sickle cell anemia, adenosine deaminase deficiency, Fragile X Syndrome, α-1-antitrypsin deficiency, Marfan syndrome, neurofibromatosis, retinoblastoma, polydactyly, phenylketonuria, Tay-Sachs disease, hemophilia A, Duchenne Becker muscular dystrophy, glucose-6-phosphate dehydrogenase deficiency or Rett syndrome.


SEQUENCE LISTING (4 sequences)

SEQ ID NO: 1 - DNA, 31 bp, Artificial Sequence
  Synthetic primer
cttttgctggccttttgctcacatgtcctgc

SEQ ID NO: 2 - DNA, 30 bp, Artificial Sequence
  Synthetic primer
gtaaggagaaaataccgcatcaggcgcccc

SEQ ID NO: 3 - DNA, 37 bp, Artificial Sequence
  Synthetic primer
tcctggccttttgctggccttttgctcacatgtcctg

SEQ ID NO: 4 - DNA, 145 bp, Artificial Sequence
  Synthetic ITR sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa